(19) United States
(12) Patent Application Publication
(10) Pub. No.: US 2022/0017570 A1
(43) Pub. Date: Jan. 20, 2022

(54) METHOD FOR PURIFYING PEGYLATED PROTEIN

(71) Applicant: Bristol-Meyers Squibb Company, Princeton, NJ (US)

(72) Inventors: Deqiang YU, Devens, MA (US); John PAGANO, Devens, MA (US); Hasin FEROZ, Devens, MA (US); Yuanli SONG, Devens, MA (US); Sanchayita GHOSE, Devens, MA (US); Zhengjian LI, Devens, MA (US)

(21) Appl. No.: 17/291,562

(22) PCT Filed: Nov. 4, 2019
(86) PCT No.: PCT/US2019/059658

§ 371 Date: May 5, 2021

Related U.S. Application Data
(60) Provisional application No. 62/756,020, filed on Nov. 5, 2018.

(51) Int. Cl. C07K 1/18; C07K 14/50; A61K 47/60

(52) U.S. Cl. CPC C07K 1/18; A61K 47/60; C07K 14/50

(57) ABSTRACT

This disclosure provides a novel method of purifying PEGylated proteins using ion exchange chromatography by loading PEGylated protein having a high concentration, e.g., at least 6 grams/liter on an ion exchange chromatography matrix, and collecting the PEGylated protein.

### CROSS-REFERENCE TO RELATED APPLICATION

This patent application claims priority to U.S. Provisional Patent Application 62/756,020, filed on Nov. 5, 2018, which is hereby incorporated by reference in its entirety.

### BACKGROUND

Chemical modification of proteins or biopharmaceuticals by covalent attachment of polyethylene glycol molecules (PEG) molecules can impart several significant advantages over the unmodified proteins or biopharmaceuticals, including prolonged half-life, enhanced aqueous solubility, reduced toxicity, reduced rate of renal clearance, and reduced immunogenicity and antigenicity of modified proteins or biopharmaceuticals. These advantages appear to be mainly due to the significantly increased molecular size (hydrodynamic radius) and surface alteration and protection (“masking”) by the neutral, chemically inert, hydrophilic PEG polymers.

One of the challenges associated with the production of PEGylated proteins is that a heterogeneous product results from the PEGylation process, including unreacted native protein, unreacted PEG, and PEGylated species with a range of PEGylation sites and varying extents of conjugation. When the PEGylated product is to be used as a therapeutic, purification of the PEGylated therapeutic molecules from undesired residual impurities is a necessity.

Several methods for purifying PEGylated proteins such as size exclusion chromatography (SEC), hydrophobic interaction chromatography, and most commonly, electrostatic interaction chromatography (ion exchange chromatography, IEC) are presently used. However, due to several factors related to the nature of PEG polymers, purifying PEGylated proteins is complicated. The PEG polymers are neutral, hydrophilic, and their solubility in aqueous solutions decreases inversely with temperature. PEGylation reaction product mixtures containing PEGs and PEGylated proteins can exhibit foaming, viscosity, and protein or polymer precipitation. Since PEGylated products are high molecular weight polymers, they tend to nonspecifically adsorb to surfaces and tend to increase the viscosity of aqueous solutions. These characteristics have forced lowering loading solution concentration for the purpose of chromatographic purification to about 1 gram/liter in order to accommodate the chromatographic media's capacity, resulting in low yields and costly purification methods.

Therefore, there is a need to develop a rapid and economical method for purifying PEGylated products that results in higher yields of pure PEGylated product.

### BRIEF SUMMARY OF THE INVENTION

The present disclosure provides an efficient method for purifying PEGylated products using ion exchange chromatography. Surprisingly, it was discovered that, in contradiction to the practiced chromatography principles, increasing PEGylated protein concentration loaded onto the separation matrix resulted in an unexpected increase in the ion exchange matrix's binding capacity and/or improved the purification yield of the PEGylated product. The method of the present disclosure provides both time and cost savings: (1) by increasing the concentration of protein loaded, the number of purifications cycles are reduced, and (2) the observed higher binding capacity of the matrix reduces the necessity of frequent cleaning and replacement of the chromatography matrix.

Therefore, in some embodiments, the present disclosure provides a method for purifying a PEGylated protein comprising loading PEGylated protein having a high concentration of at least 6 grams/liter on an ion exchange chromatography matrix, and collecting the PEGylated protein.

In some embodiments, the present disclosure provides a method for purifying a PEGylated protein wherein loading of the PEGylated protein having a high concentration results in an increase in the yield of the collected PEGylated protein compared to the yield of the collected PEGylated protein loaded at a concentration of 1 g/L.

In some embodiments, the present disclosure provides a method for purifying a PEGylated protein wherein the yield of the collected PEGylated protein is increased at least about 1.5 fold, at least about 2 fold, about 3 fold, about 4 fold, about 5 fold, about 6 fold, about 7 fold, about 8 fold, about 9 fold, about 10 fold, about 20 fold, about 30 fold, or about 40 fold.

In some embodiments, the present disclosure provides a method for purifying a PEGylated protein wherein the loading of the PEGylated protein having a high concentration results in an increase of the ion exchange matrix's loading capacity compared to the ion exchange matrix's loading capacity when PEGylated protein is loaded at a concentration of 1 g/L.

In some embodiments, the present disclosure provides a method for purifying PEGylated protein wherein the loading capacity of the ion exchange matrix is increased from 6 g to about 7 g, about 8 g, about 9 g, about 10 g, about 11 g, about 12 g, about 13 g, about 14 g, about 15 g, about 16 g, about 17 g, about 18 g, about 19 g, or about 20 g of PEGylated protein/L of matrix.

In some embodiments, the present disclosure provides a method for purifying PEGylated protein wherein loading of the PEGylated protein having a high concentration results in an increase of the ion exchange matrix's binding capacity, compared to the ion exchange matrix's binding capacity when PEGylated protein is loaded at a concentration of 1 g/L.

In some embodiments, the present disclosure provides a method for purifying PEGylated protein wherein the binding capacity of the ion exchange matrix is increased about 1.1 fold, about 1.2 fold, about 1.3 fold, about 1.5 fold, about 1.6 fold, about 1.7 fold, about 1.8 fold, about 1.9 fold, about 2 fold, about 3 fold, about 4 fold, about 5 fold, about 6 fold, about 7 fold, about 8 fold, about 9 fold, about 10 fold, about 15 fold, about 20 fold, or about 30 fold compared to the ion exchange matrix's loading capacity when PEGylated protein is loaded at a concentration of 1 g/L.

In some embodiments, the present disclosure provides a method for purifying PEGylated protein wherein the binding capacity of the ion exchange chromatography matrix is at least 7 g, at least 7.5 g, at least 8 g, at least 8.5 g, at least 9 g, at least 9.5 g, at least 10 g, at least 10.5 g, at least 11 g, at least 11.5 g, at least 12 g, at least 12.5 g, at least 13 g, at least 13.5 g, at least 14 g, at least 14.5 g, at last 15 g, at least 15.5 g, at least 16 g, at least 16.5 g, or at least 17 g of PEGylated protein/L of matrix.

In some embodiments, the present disclosure provides a method for purifying PEGylated protein wherein the collected PEGylated protein is at least about 20% pure, at least about 25% pure, at least about 30% pure, at least about 35% pure, at least about 40% pure, at least about 45% pure, at least about 50% pure, at least about 55% pure, at least about 60% pure, at least about 65% pure, at least about 70% pure, at least about 75% pure, at least about 80% pure, at least about 85% pure, at least about 90% pure, at least about 95% pure, or at least about 98% pure.

In some embodiments, the present disclosure provides a method for purifying PEGylated protein wherein UV interference during ion exchange chromatography is reduced at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least 98%.

In some embodiments, the present disclosure provides a method for purifying PEGylated protein wherein the loaded PEGylated protein has been concentrated without a catalyst prior to the loading.

In some embodiments, the present disclosure provides a method for purifying PEGylated protein wherein the loaded PEGylated protein has been concentrated by a tangential flow filtration prior to the loading.

In some embodiments, the present disclosure provides a method for purifying PEGylated protein wherein the PEGylated protein loaded at a high concentration has a concentration of at least about 7 g/L, at least about 8 g/L, at least about 9 g/L, at least about 10 g/L, at least about 11 g/L, at least about 12 g/L, at least about 13 g/L, at least about 14 g/L, at least about 15 g/L, at least about 20 g/L, at least about 25 g/L, at least about 30 g/L, at least about 35 g/L, at least about 40 g/L, at least about 45 g/L, at least about 50 g/L, at least about 55 g/L, or at least about 60 g/L.

In some embodiments, the present disclosure provides a method for purifying PEGylated protein wherein the PEGylated protein loaded at a high concentration has a concentration of about 10 g/L, about 15 g/L, about 20 g/L, about 25 g/L, about 30 g/L, about 35 g/L, about 40 g/L, about 45 g/L, about 50, about 55, or about 60 g/L.

In some embodiments, the present disclosure provides a method for purifying PEGylated protein wherein the PEGylated protein loaded at a high concentration has a concentration of about 30 g/L.

In some embodiments, the present disclosure provides a method for purifying PEGylated protein wherein the yield of the PEGylated protein is at least 5%, at least 10%, at least 15%, at least 20%, at least 25%, at least 30%, at least 35%, at least 40%, at least 45%, at least 50%, at least 55%, at least 60%, at least 65%, at least 65%, at least 70%, at least 75%, at least 80%, at least 85%, at least 90%, at least 95%, at least 98%, or at least 99%.

In some embodiments, the present disclosure provides a method for purifying PEGylated protein, further comprising washing the matrix using a wash buffer.

In some embodiments, the present disclosure provides a method for purifying PEGylated protein, further comprising eluting the PEGylated protein using an elution buffer.

In some embodiments, the present disclosure provides a method for purifying PEGylated protein wherein the ion exchange chromatography is a cation exchange chromatography.

In some embodiments, the present disclosure provides a method for purifying PEGylated protein, wherein the ion exchange chromatography comprises a CEX resin selected from the group consisting of Poros HS, Poros XS, carboxy-methyl-cellulose, BAKERBOND ABX™, sulphopropyl immobilized on agarose and sulphonyl immobilized on agarose, MonoS, MiniS, Source 15S, 30S, SP SEPHAROSE™, CM SEPHAROSE™, BAKERBOND Carboxy-Sulfon, WP CBX, WP Sulfonic, Hydrocell CM, Hydrocel SP, UNOsphere S, Macro-Prep High S, Macro-Prep CM, Ceramic HyperD S, Ceramic HyperD CM, Ceramic HyperD Z, Trisacryl M CM, Trisacryl LS CM, Trisacryl M SP, Trisacryl LS SP, Spherodex LS SP, DOWEX Fine Mesh Strong Acid Cation Resin, DOWEX MAC-3, Matrex Cellufine C500, Matrex Cellufine C200, Fractogel EMD SO3-, Fractogel EMD SE, Fractogel EMD COO—, Amberlite Weak and Strong Cation Exchangers, Diaion Weak and Strong Cation Exchangers, TSK Gel SP—SPW-HR, TSK Gel SP-SPW, Toyopearl CM (650S, 650M, 650C), Toyopearl SP (650S, 650M, 650C), CM (23, 32, 52), SE(52, 53), P11, Express-Ion C and Express-Ion S, and any combination thereof.

In some embodiments, the present disclosure provides a method for purifying PEGylated protein wherein the ion exchange chromatography is an anion exchange chromatography.

In some embodiments, the present disclosure provides a method for purifying PEGylated protein wherein the ion exchange chromatography comprises a AEX resin selected from the group consisting of POROS HQ, POROS XQ, Q SEPHAROSE™ Fast Flow, DEAE SEPHAROSE™ Fast Flow, SARTOBIND® Q, ANX SEPHAROSE™ 4 Fast Flow (high sub), Q SEPHAROSE™ XL, Q SEPHAROSE™ big beads, DEAE Sephadex A-25, DEAE Sephadex A-50, QAE Sephadex A-25, QAE Sephadex A-50, Q SEPHAROSE™ high performance, Q SEPHAROSE™ XL, Sourse 15Q, Sourse 30Q, Resourse Q, Capto Q, Capto DEAE, Mono Q, Toyopearl Super Q, Toyopearl DEAE, Toyopearl QAE, Toyopearl Q, Toyopearl GigaCap Q, TS gel SuperQ, TS gel DEAE, Fractogel EMD TMAE, Fractogel EMD TMAE HiCap, Fractogel EMD DEAE, Fractogel EMD DMAE, Macroprep High Q, Macro-prep-DEAE, Unosphere Q, Nuvia Q, PORGS PI, DEAE Ceramic HyperD, Q Ceramic HyperD, and any combination thereof.

In some embodiments, the present disclosure provides a method for purifying PEGylated protein wherein the PEGylated protein has a molecular weight of at least about 5 kDa, at least about 10 kDa, at least about 15 kDa, at least about 20 kDa, at least about 25 kDa, at least about 30 kDa, at least about 35 kDa, at least about 40 kDa, at least about 45 kDa, at least about 50 kDa, at least about 55 kDa, at least about 60 kDa, at least about 75 kDa, at least about 80 kDa, at least about 85 kDa, at least about 90 kDa, at least about 95 kDa, at least about 100 kDa, at least about 105 kDa, at least about 110 kDa, at least about 115 kDa, at least about 120 kDa, at least about 125 kDa, at least about 130 kDa, at least about 135 kDa, at least about 140 kDa, at least about 145 kDa, at least about 150 kDa, at least about 155 kDa, at least about 160 kDa, at least about 165 kDa, at least about 170 kDa, at least about 175 kDa, at least about 180 kDa, at least about 185 kDa, at least about 190 kDa, at least about 195 kDa, at least about 200 kDa, at least about 300 kDa, at least about 350 kDa, at least about 400 kDa, at least about 450 kDa, at least about 500 kDa, at least about 550 kDa, or at least about 600 kDa.

In some embodiments, the present disclosure provides a method for purifying PEGylated protein wherein the PEGylated protein is a wild-type protein, a mutant, a derivative, a variant, or a fragment that has been PEGylated.

In some embodiments, the present disclosure provides a method for purifying PEGylated protein wherein the PEGylated protein is a naturally occurring or recombinantly produced protein that has been PEGylated.

In some embodiments, the present disclosure provides a method for purifying PEGylated protein wherein the PEGylated protein is an antibody or a fusion protein.

In some embodiments, the present disclosure provides a method for purifying PEGylated protein wherein the PEGylated protein is a cytokine, a clotting factor, a hormone, a cell surface receptor, a growth factor, or any combination thereof.

In some embodiments, the present disclosure provides a method for purifying PEGylated protein wherein the PEGylated protein is Fibroblast Growth Factor 21 (FGF21), Interleukin 2, Factor VIII, recombinant phenylalanine ammonia-lyase, Pegvaliase, Adynovate, an interferon (e.g., Interferon Beta-1a (e.g., Plegridy)), naloxol (e.g., Naloxegol), Peginesatide, Certolizumab pegol, erythropoietin (e.g., methoxy polyethylene glycol-epoetin beta), Pegaptanib, a recombinant methionyl human granulocyte colony-stimulating factor, Pegfilgrastim, a human growth hormone antagonist (e.g., Pegvisomant), interferon alpha, (e.g., Peginterferon alfa-2a or Peginterferon alfa-2b), L-asparaginase (e.g., Pegaspargase), adenosine deaminase (e.g., Pegademase bovine), or doxorubicin.

In some embodiments, the present disclosure provides a method for purifying PEGylated protein wherein the PEGylated protein is FGF21.

In some embodiments, the present disclosure provides a method for purifying PEGylated protein wherein the PEGylated protein comprises a PEGylation moiety.

In some embodiments, the present disclosure provides a method for purifying PEGylated protein wherein the PEGylation moiety is linear, branched, mono-PEGylated, random PEGylated, and multiple PEGylated (PEGmers).

In some embodiments, the present disclosure provides a method for purifying PEGylated protein wherein the PEGylation moiety is at least about 1 kDa, at least about 2 kDa, at least about 3 kDa, at least about 4 kDa, at least about 5 kDa, at least about 6 kDa, at least about 7 kDa, at least about 8 kDa, at least about 9 kDa, at least about 10 kDa, at least about 11 kDa, at least about 12 kDa, at least about 13 kDa, at least about 14 kDa, at least about 15 kDa, at least about 16 kDa, at least about 17 kDa, at least about 18 kDa, at least about 19 kDa, at least about 20 kDa, at least about 21 kDa, at least about 22 kDa, at least about 23 kDa, at least about 24 kDa, at least about 25 kDa, at least about 30 kDa, at least about 40 kDa, at least about 50 kDa, at least about 55 kDa, at least about 60 kDa, at least about 65 kDa, at least about 70 kDa, at least about 75 kDa, at least about 80 kDa, at least about 90 kDa, at least about 95 kDa, or at least about 100 kDa.

In some embodiments, the present disclosure provides a method for purifying PEGylated protein wherein the PEGylation moiety is about 30 kDa.

In some embodiments, the present disclosure provides a purified PEGylated protein using the method of the present disclosure.

### BRIEF DESCRIPTION OF THE DRAWINGS/FIGURES

FIG. 1: Impact of protein concentration on PEG-protein size (Rh: radius hydrodynamic in nanometers; DLS: Dynamic Light Scattering) and binding capacity. PEGylated protein concentration increased from 1 g/L to 30 g/L, Rh of PEGylated protein decreased from 6.1 nm to 2.7 nm while binding capacity increased from 5.1 g/L resin to 12.8 g/L resin. AEX: anion exchange chromatography.

FIG. 2: Comparison of loading concentration and of PEGylated protein and binding capacity of IEC matrix under current protocols (31× dilution of PEGylated reaction products; left side) and under the new method of the present disclosure (center and right side): for example no dilution, concentration by TFF (tangential flow filtration; with 10 kDa and 30 kDa pore size as indicated), and loading on IEC matrix. 4-ABH: 4-aminobenzoic hydrazide (catalyst of PEGylation reaction).

### DETAILED DESCRIPTION OF THE INVENTION

The present disclosure provides an effective method for purifying the desired PEGylated target from undesired impurities. Going against the teaching in the art, the method of the present disclosure includes loading onto an ion exchange matrix a high concentration of the PEGylated protein, of at least 6 g/L, instead of a diluted solution of lower than 6 g/L, e.g., 1 g/L. It has been surprisingly found that the high concentration of the loaded PEGylated protein solution increased the binding capacity and loading capacity of the chromatography matrix, and produced a high yield of the purified protein. The method of the present disclosure saves time, labor, and expenses by reducing the number of purification cycles required, which in turn reduces the need for cleaning and replacing costly chromatography matrix, in order to obtain the desired PEGylated protein.

PEGylated proteins are formed from the chemical attachment of a PEG chain to the native protein using a variety of different chemical reagents. In certain embodiments, the present disclosure provides a method of purifying a PEGylated protein of interest from a mixture which comprises the PEGylated protein of interest and one or more contaminants. Possible contaminants include unreacted PEG, unreacted native protein, reaction catalyst, host cell proteins (HCP), high molecular weight species (HMWs), low molecular weight species (LMWs), and DNA. The present disclosure also provides a method of purifying the desired PEGylated target from impurities in the solution by loading onto a chromatography matrix or ion exchange matrix, a solution with a high concentration of PEGylated protein of at least 6 grams per liter, and collecting the target PEGylated product.

### I. Definitions

In order that the present disclosure may be more readily understood, certain terms are first defined.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

As used herein, the term “protein” or “protein of interest” is used in its broadest sense to include any protein (either natural or recombinant), present in a mixture, for which purification is desired. Such proteins of interest include, without limitation, enzymes, hormones, growth factors, cytokines, immunoglobulins (e.g., antibodies), and/or any fusion proteins, and derivatives and portions thereof.

The terms “purifying,” “separating,” or “isolating,” as used interchangeably herein, refer to increasing the degree of purity of a protein of interest from a composition or sample comprising the protein of interest and one or more impurities. Typically, the degree of purity of the protein of interest is increased by removing (completely or partially) at least one impurity from the composition.

The term “buffer” as used herein, refers to a substance which, by its presence in solution, increases the amount of acid or alkali that must be added to cause unit change in pH. A buffered solution resists changes in pH by the action of its acid-base conjugate components. Buffered solutions for use with biological reagents are generally capable of maintaining a constant concentration of hydrogen ions such that the pH of the solution is within a physiological range. Traditional buffer components include, but are not limited to, organic and inorganic salts, acids and bases.

The term “chromatography” refers to any kind of technique which separates a protein of interest (e.g., a PEGylated protein) from other molecules (e.g., contaminants) present in a mixture. Usually, the protein of interest is separated from other molecules (e.g., contaminants) as a result of differences in rates at which the individual molecules of the mixture migrate through a stationary medium under the influence of a moving phase, or in bind and elute processes. The term “matrix” or “chromatography matrix” are used interchangeably herein and refer to any kind of sorbent, resin or solid phase which in a separation process separates a protein of interest (e.g., an Fc region containing protein such as an immunoglobulin) from other molecules present in a mixture. Non-limiting examples include particulate, monolithic or fibrous resins as well as membranes that can be put in columns or cartridges. Examples of materials for forming the matrix include polysaccharides (such as agarose and cellulose); and other mechanically stable matrices such as silica (e.g. controlled pore glass), poly(styrenedivinyl)benzene, polyacrylamide, ceramic particles and derivatives of any of the above. Examples for typical matrix types suitable for the method of the present disclosure are cation exchange resins, affinity resins, anion exchange resins or mixed mode resins. A “ligand” is a functional group that is attached to the chromatography matrix and that determines the binding properties of the matrix. Examples of “ligands” include, but are not limited to, ion exchange groups, hydrophobic interaction groups, hydrophilic interaction groups, thiophilic interactions groups, metal affinity groups, affinity groups, bioaffinity groups, and mixed mode groups (combinations of the aforementioned). Some preferred ligands that can be used herein include, but are not limited to, strong cation exchange groups, such as sulphopropyl, sulfonic acid; strong anion exchange groups, such as trimethylammonium chloride; weak cation exchange groups, such as carboxylic acid; weak anion exchange groups, such as N5N diethylamino or DEAE; hydrophobic interaction groups, such as phenyl, butyl, propyl, hexyl; and affinity groups, such as Protein A, Protein G, and Protein L.

The term “chromatography column” or “column” in connection with chromatography as used herein, refers to a container, frequently in the form of a cylinder or a hollow pillar which is filled with the chromatography matrix or resin. The chromatography matrix or resin is the material which provides the physical and/or chemical properties that are employed for purification.

The terms “ion-exchange” and “ion-exchange chromatography” refer to a chromatographic process in which an ionizable solute of interest (e.g., a protein of interest in a mixture) interacts with an oppositely charged ligand linked (e.g., by covalent attachment) to a solid phase ion exchange material under appropriate conditions of pH and conductivity, such that the solute of interest interacts non-specifically with the charged compound more or less than the solute impurities or contaminants in the mixture. The contaminating solutes in the mixture can be washed from a column of the ion exchange material or are bound to or excluded from the resin, faster or slower than the solute of interest. “Ion-exchange chromatography” specifically includes cation exchange (CEX), anion exchange (AEX), and mixed mode chromatography. Ion exchange chromatography is interchangeably referred herein as IEC and IEX.

A “cation exchange resin” or “cation exchange membrane” refers to a solid phase which is negatively charged, and which has free cations for exchange with cations in an aqueous solution passed over or through the solid phase. Any negatively charged ligand attached to the solid phase suitable to form the cation exchange resin can be used, e.g., a carboxylate, sulfonate and others as described below. Commercially available cation exchange resins include, but are not limited to, for example, those having a sulfonate based group (e.g., MonoS, MiniS, Source 15S and 30S, SP SEPHAROSE® Fast Flow, SP SEPHAROSE® High Performance, Capto S, Capto SP ImpRes from GE Healthcare, TOYOPEARL® SP-650S and SP-650M from Tosoh, MACRO-PREP® High S from BioRad, Ceramic HyperD S, TRISACRYL® M and LS SP and Spherodex LS SP from Pall Technologies); a sulfoethyl based group (e.g., FRACTOGEL® SE, from EMD, POROS® S-10 and S-20 from Applied Biosystems); a sulphopropyl based group (e.g., TSK Gel SP 5PW and SP-5PW-HR from Tosoh, POROS® HS-20, HS 50, and POROS® XS from Life Technologies); a sulfoisobutyl based group (e.g., FRACTOGEL® EMD SO₃⁻ from EMD); a sulfoxyethyl based group (e.g., SE52, SE53 and Express-Ion S from Whatman), a carboxymethyl based group (e.g., CM SEPHAROSE® Fast Flow from GE Healthcare, Hydrocell CM from Biochrom Labs Inc., MACRO-PREP® CM from BioRad, Ceramic HyperD CM, TRISACRYL® M CM, TRISACRYL® LS CM, from Pall Technologies, Matrx CELLUFINE® C500 and C200 from Millipore, CM52, CM32, CM23 and Express-Ion C from Whatman, TOYOPEARL® CM-650S, CM-650M and CM-650C from Tosoh); sulfonic and carboxylic acid based groups (e.g., BAKERBOND® Carboxy-Sulfon from J. T. Baker); a carboxylic acid based group (e.g., WP CBX from J. T Baker, DOWEX®. MAC-3 from Dow Liquid Separations, AMBERLITE® Weak Cation Exchangers, DOWEX® Weak Cation Exchanger, and DIAION® Weak Cation Exchangers from Sigma-Aldrich and FRACTOGEL® EMD COO— from EMD); a sulfonic acid based group (e.g., Hydrocell SP from Biochrom Labs Inc., DOWEX® Fine Mesh Strong Acid Cation Resin from Dow Liquid Separations, UNOsphere S, WP Sulfonic from J. T. Baker, SARTOBIND® S membrane from Sartorius, AMBERLITE® Strong Cation Exchangers, DOWEX® Strong Cation and DIAION® Strong Cation Exchanger from Sigma-Aldrich); or a orthophosphate based group (e.g., P11 from Whatman). Other cation exchange resins include carboxy-methyl-cellulose, BAKERBOND ABX™, Ceramic HyperD Z, Matrex Cellufine C500, Matrex Cellufine C200.

An “anion exchange resin” or “anion exchange membrane” refers to a solid phase which is positively charged, thus having one or more positively charged ligands attached thereto. Any positively charged ligand attached to the solid phase suitable to form the anionic exchange resin can be used, such as quaternary amino groups. Commercially available anion exchange resins include DEAE cellulose, POROS® PI 20, PI 50, HQ 10, HQ 20, HQ 50, D 50 from Applied Biosystems, SARTOBIND® Q from Sartorius, MonoQ, MiniQ, Source 15Q and 30Q, Q, DEAE and ANX SEPHAROSE® Fast Flow, Q SEPHAROSE® High Performance, QAE SEPHADEX® and FAST Q SEPHAROSE® (GE Healthcare), WP PEI, WP DEAM, WP QUAT from J. T. Baker, Hydrocell DEAE and Hydrocell QA from Biochrom Labs Inc., UNOsphere Q, MACRO-PREP®. DEAE and MACRO-PREP® High Q from Biorad, Ceramic HyperD Q, ceramic HyperD DEAE, TRISACRYL® M and LS DEAE, Spherodex LS DEAE, QMA SPHEROSIL® LS, QMA SPHEROSIL®. M and MUSTANG® Q from Pall Technologies, DOWEX® Fine Mesh Strong Base Type I and Type II Anion Resins and DOWEX® MONOSPHER E 77, weak base anion from Dow Liquid Separations, INTERCEPT® Q membrane, Matrex CELLUFINE® A200, A500, Q500, and Q800, from Millipore, FRACTOGEL® EMD TMAE, FRACTOGEL® EMD DEAE and FRACTOGEL® EMD DMAE from EMD, AMBERLITE® weak strong anion exchangers type I and II, DOWEX® weak and strong anion exchangers type I and II, DIAION® weak and strong anion exchangers type I and II, DUOLITE® from Sigma-Aldrich, TSK gel Q and DEAE 5PW and 5PW-HR, TOYOPEARL® SuperQ-650S, 650M and 650C, QAE-550C and 650S, DEAE-650M and 650C from Tosoh, QA52, DE23, DE32, DE51, DE52, DE53, Express-Ion D or Express-Ion Q from Whatman, and SARTOBIND® Q (Sartorius Corporation, New York, USA). Other anion exchange resins include POROS XQ, SARTOBIND® Q, Q SEPHAROSE™ XL, Q SEPHAROSE™ big beads, DEAE Sephadex A-25, DEAE Sephadex A-50, QAE Sephadex A-25, QAE Sephadex A-50, Q SEPHAROSE™ high performance, Q SEPHAROSE™ XL, Resource Q, Capto Q, Capto DEAE, Toyopearl GigaCap Q, Fractogel EMD TMAE HiCap, Nuvia Q, or PORGS PI.

As used herein the term “contaminant” is used in its broadest sense to cover any undesired component or compound within a mixture. In cell cultures, cell lysates, or clarified bulk (e.g., clarified cell culture supernatant), contaminants include, for example, host cell nucleic acids (e.g., DNA) and host cell proteins present in a cell culture medium. Host cell contaminant proteins include, without limitation, those naturally or recombinantly produced by the host cell, as well as proteins related to or derived from the protein of interest (e.g., proteolytic fragments) and other process related contaminants. In certain embodiments, the contaminant precipitate is separated from the cell culture using another means, such as centrifugation, sterile filtration, depth filtration and tangential flow filtration.

The term “antibody” refers, in some embodiments, to a protein comprising at least two heavy (H) chains and two light (L) chains inter-connected by disulfide bonds. Each heavy chain is comprised of a heavy chain variable region (abbreviated herein as VH) and a heavy chain constant region (abbreviated herein as CH). In some antibodies, e.g., naturally-occurring IgG antibodies, the heavy chain constant region is comprised of a hinge and three domains, CH1, CH2 and CH3. In some antibodies, e.g., naturally-occurring IgG antibodies, each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. A heavy chain may have the C-terminal lysine or not. The term “antibody” can include a bispecific antibody or a multispecific antibody.

An “IgG antibody”, e.g., a human IgG1, IgG2, IgG3 and IgG4 antibody, as used herein has, in some embodiments, the structure of a naturally-occurring IgG antibody, i.e., it has the same number of heavy and light chains and disulfide bonds as a naturally-occurring IgG antibody of the same subclass. For example, an IgG1, IgG2, IgG3 or IgG4 antibody may consist of two heavy chains (HCs) and two light chains (LCs), wherein the two HCs and LCs are linked by the same number and location of disulfide bridges that occur in naturally-occurring IgG1, IgG2, IgG3 and IgG4 antibodies, respectively (unless the antibody has been mutated to modify the disulfide bridges).

An immunoglobulin can be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. Immunoglobulins, e.g., IgG1, exist in several allotypes, which differ from each other in at most a few amino acids. “Antibody” includes, by way of example, both naturally-occurring and non-naturally-occurring antibodies; monoclonal and polyclonal antibodies; chimeric and humanized antibodies; human and nonhuman antibodies and wholly synthetic antibodies.

The term “antigen-binding portion” of an antibody, as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment (fragment from papain cleavage) or a similar monovalent fragment consisting of the VL, VH, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; (vi) an isolated complementarity determining region (CDR) and (vii) a combination of two or more isolated CDRs which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences.

As used herein, “isotype” refers to the antibody class (e.g., IgG1, IgG2, IgG3, IgG4, IgM, IgA1, IgA2, IgD, and IgE antibody) that is encoded by the heavy chain constant region genes.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

As used herein, the term “polypeptide” refers to a molecule composed of monomers (amino acids) linearly linked by amide bonds (also known as peptide bonds). The term “polypeptide” refers to any chain or chains of two or more amino acids, and does not refer to a specific length of the product. As used herein the term “protein” is intended to encompass a molecule comprised of one or more polypeptides, which can in some instances be associated by bonds other than amide bonds. On the other hand, a protein can also be a single polypeptide chain. In this latter instance the single polypeptide chain can in some instances comprise two or more polypeptide subunits fused together to form a protein. The terms “polypeptide” and “protein” also refer to the products of post-expression modifications, including without limitation glycosylation, acetylation, phosphorylation, amidation, derivatization by known protecting/blocking groups, proteolytic cleavage, or modification by non-naturally occurring amino acids. A polypeptide or protein can be derived from a natural biological source or produced by recombinant technology, but is not necessarily translated from a designated nucleic acid sequence. It can be generated in any manner, including by chemical synthesis.

The terms “polynucleotide” or “nucleotide” as used herein are intended to encompass a singular nucleic acid as well as plural nucleic acids, and refers to an isolated nucleic acid molecule or construct, e.g., messenger RNA (mRNA), complementary DNA (cDNA), or plasmid DNA (pDNA). In certain aspects, a polynucleotide comprises a conventional phosphodiester bond or a non-conventional bond (e.g., an amide bond, such as found in peptide nucleic acids (PNA)).

The term “nucleic acid” refers to any one or more nucleic acid segments, e.g., DNA, cDNA, or RNA fragments, present in a polynucleotide. When applied to a nucleic acid or polynucleotide, the term “isolated” refers to a nucleic acid molecule, DNA or RNA, which has been removed from its native environment, for example, a recombinant polynucleotide encoding an antigen binding protein contained in a vector is considered isolated for the purposes of the present disclosure. Further examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present disclosure. Isolated polynucleotides or nucleic acids according to the present disclosure further include such molecules produced synthetically. In addition, a polynucleotide or a nucleic acid can include regulatory elements such as promoters, enhancers, ribosome binding sites, or transcription termination signals.

The term “isoelectric point” or “pI” of a protein refers to a measure of the pH of a solution in which a protein carries no net charge. When a protein is found at a pH equivalent to its pI, it will carry globally neutral net electric charge. Proteins that have a pI lower than the pH of its solution will carry a net negative charge. Likewise, proteins that have a pI higher than the pH of its solution will carry a net positive charge.

The term “loading buffer” refers to the buffer used to prepare and load a mixture or sample into the chromatography unit.

The term “chase buffer” refers to the buffer used subsequent to the loading buffer, in order to drive the mixture or sample through the chromatographic process.

The term “HMW Species” refers to any one or more unwanted proteins present in a mixture. High molecular weight species can include dimers, trimers, tetramers, or other multimers. These species are often considered product related impurities, and can either be covalently or non-covalently linked, and can also, for example, consist of misfolded monomers in which hydrophobic amino acid residues are exposed to a polar solvent, and can cause aggregation.

The term “LMW Species” refers to any one or more unwanted species present in a mixture. Low molecular weight species are often considered product related impurities, and can include clipped species, or half molecules for compounds intended to be dimeric (such as monoclonal antibodies).

The term “Host Cell Proteins” or HCP refers to the undesirable proteins generated by a host cell unrelated to the production of the intended protein of interest. Undesirable host cell proteins can be secreted into the upstream cell culture supernatant. Undesirable host cell proteins can also be released during cell lysis. The cells used for upstream cell culture require proteins for growth, transcription, and protein synthesis, and these unrelated proteins are undesirable in a final drug product.

The term “loading” and grammatical equivalents thereof as used within this application denotes a step of a purification methods in which a solution containing a substance of interest to be purified is brought in contact with a stationary phase. This denotes that that a) the solution is added to a chromatographic device in which the stationary phase is located, or b) that a stationary phase is added to the solution. In case a) the solution containing the substance of interest to be purified passes through the stationary phase allowing for an interaction between the stationary phase and the substances in solution. Depending on the conditions, such as, e.g., pH, conductivity, salt concentration, temperature, and/or flow rate, some substances of the solution are bound to the stationary phase and thus are removed from the solution. Other substances remain in solution. The substances remaining in solution can be found in the flow-through. The “flow-through” denotes the solution obtained after the passage of the chromatographic device, which may either be the loaded solution containing the substance of interest or the buffer, which is used to flush the column or to cause elution of one or more substances bound to the stationary phase. In one embodiment the chromatographic device is a column, or a cassette. The substance of interest can be recovered or “collected” from the solution after the purification step by methods familiar to a person of skill in the art, such as, e.g., precipitation, salting out, ultrafiltration, diafiltration, lyophilization, affinity chromatography, or solvent volume reduction to obtain the substance of interest in substantially homogeneous form. In case b) the stationary phase is added, e.g., as a solid, to the solution containing the substance of interest to be purified allowing for an interaction between the stationary phase and the substances in solution. After the interaction the stationary phase is removed, e.g., by filtration, and the substance of interest is either bound to the stationary phase and removed therewith from the solution or not bound to the stationary phase and remains in the solution.

The term “under conditions suitable for binding” and grammatical equivalents thereof as used within this application denotes that a substance of interest, e.g., PEGylated protein, binds to a stationary phase when brought in contact with it, e.g., an ion exchange material. This does not necessarily mean that 100% of the substance of interest is bound but essentially 100% of the substance of interest is bound, i.e., at least 50% of the substance of interest is bound, more preferably at least 75% of the substance of interest is bound, even more preferably at least 85% of the substance of interest is bound, and especially preferably more than 95% of the substance of interest is bound to the stationary phase.

The term “buffered” as used within this application denotes a solution in which changes of pH due to the addition or release of acidic or basic substances is leveled by a buffer substance. Any buffer substance resulting in such an effect can be used. In some embodiments, pharmaceutically acceptable buffer substances are used, such as, e.g., phosphoric acid or salts thereof, acetic acid or salts thereof, citric acid or salts thereof, morpholine, 2-(N-morpholino) ethanesulfonic acid or salts thereof, histidine or salts thereof, glycine or salts thereof, or tris(hydroxymethyl)aminomethane (TRIS) or salts thereof. In one embodiment phosphoric acid or salts thereof, or acetic acid or salts thereof, or citric acid or salts thereof, or histidine or salts thereof are used as the buffer substance. Optionally the buffered solution can comprise an additional salt, such as, e.g., sodium chloride, sodium sulphate, potassium chloride, potassium sulfate, sodium citrate, or potassium citrate.

General chromatographic methods and their use are known to a person skilled in the art. See for example, Chromatography, 5^{th} edition, Part A: Fundamentals and Techniques, Heftmann, E. (ed), Elsevier Science Publishing Company, New York, (1992); Advanced Chromatographic and Electromigration Methods in Biosciences, Deyl, Z. (ed.), Elsevier Science BV, Amsterdam, The Netherlands, (1998); Chromatography Today, Poole, C. F., and Poole, S. K., Elsevier Science Publishing Company, New York, (1991); Scopes, Protein Purification Principles and Practice (1982); Sambrook, J., et al. (ed), Molecular Cloning: A Laboratory Manual, Second Edition, Cold Spring Harbor Laboratory Press, Cold Spring Harbor, N.Y., 1989; or Current Protocols in Molecular Biology, Ausubel, F. M., et al. (eds), John Wiley & Sons, Inc., New York.

“PEG” or “PEG group” according to the disclosure means a residue containing poly(ethylene glycol) as an essential part. Such a PEG can contain further chemical groups which are necessary for binding, i.e., conjugation, reactions, which result from the chemical synthesis of the molecule, or which is a spacer for optimal distance of parts of the molecule. In addition, such a PEG can consist of one or more PEG side-chains, which are linked together. PEGs with more than one PEG chain are called multiarmed or branched PEGs. Branched PEGs can be prepared, for example, by the addition of polyethylene oxide to various polyols, including glycerol, pentaerythriol, and sorbitol.

The term “PEGylation” means a covalent linkage of a poly (ethylene glycol) residue at the N-terminus of the polypeptide and/or an internal amino acid, e.g., a lysine residue. PEGylation of proteins is widely known in the state of the art and is reviewed by, for example, Bonora G., Diroli S. Reactive PEGs for protein conjugation. In: Veronese F M, ed. PEGylated Protein Drugs: basic Science and Clinical Applications. Basel: Birkhauser; 2009:33-45. See also, Veronese, F. M., Biomaterials 22 (2001) 405-417. PEG can be linked using different functional groups, and polyethylene glycols with different molecular weight and linear and branched PEGs, as well as different linking groups, are known in the art (see also Francis, G. E., et al., Int. J. Hematol. 68 (1998) 1-18; Delgado, C., et al., Crit. Rev. Ther. Drug Carrier Systems 9 (1992) 249-304). PEGylation can be performed in aqueous solution with PEGylation reagents as described by using NETS-activated linear or branched PEG molecules. PEGylation can also be performed at the solid phase according to Lu, Y., et al., Reactive Polymers 22 (1994) 221-229.

Suitable PEG derivatives are activated PEG molecules with an average molecular weight of from about 2 kDa to about 40 kDa, in one embodiment from about 20 to about 40 kDa, preferably about 30 kDa to about 35 kDa. The PEG derivative is in one embodiment a linear or a branched PEG. A wide variety of PEG derivatives suitable for use in the preparation of PEG-protein and PEG-peptide conjugates can be obtained from Shearwater Polymers (Huntsville, Ala., U.S.A.; www.nektar.com).

Activated PEG derivatives are known in the art and are described in, for example, Morpurgo, M., et al., J. Bioconjug. Chem. 7 (1996) 363-368, for PEG-vinylsulfone. Linear chain and branched chain PEG species are suitable for the preparation of the PEGylated fragments. Examples of reactive PEG reagents are iodo-acetyl-methoxy-PEG, or methoxy-PEG-vinylsulfone (m is preferably an integer from about 450 to about 900 and R is a to C₆-alkyl, linear or branched, having one to six carbon atoms such as methyl, ethyl, isopropyl, etc. The use of these iodo-activated substances is known in the art and described, e.g. by Hermanson, G. T., in Bioconjugate Techniques, Academic Press, San Diego (1996) p. 147-148.

### II. Methods of Purification

The PEGylation of a protein normally results in a mixture of different compounds, such as poly-PEGylated protein, mono-PEGylated protein, non-PEGylated protein, hydrolysis products of the activated PEG ester, e.g., the free PEGylated acid, as well as hydrolysis products of the protein itself, as well PEGylation reaction catalysts. In order to obtain the desired PEGylated product, these substances have to be separated and the PEGylated protein of interest has to be purified.

Therefore, in one aspect, the current disclosure provides a method for obtaining a PEGylated protein in substantially purified form comprising loading a solution of high concentration of PEGylated protein onto ion exchange material, and recovering or collecting the purified PEGylated protein. In some embodiments, the present method is directed to a method for purifying a PEGylated protein, comprising loading PEGylated protein having a high concentration of at least about 6 grams/liter (g/L), e.g., at least about 10 g/L, at least about 15 g/L, or at least about 30 g/L, on an ion exchange chromatography matrix, and collecting the PEGylated protein.

As an example of a chromatography process comprising the purification of the present disclosure, the mixture of mono-PEGylated or poly-PEGylated protein is applied at a protein concentration of at least about 6 g/L to the ion exchange chromatography column in an aqueous buffered solution. In one aspect, the mixture is concentrated using tangential flow filtration and the catalyst is removed in order to reduce UV interference and protein concentration determination. In a further embodiment, prior to and after the application the first column is washed with the same buffer solution. In the recovery step of the polypeptide bound to the ion exchange material, the ionic strength, i.e., the conductivity, of the buffer/solution passing through the ion exchange column is increased. This can be accomplished either by an increased buffer salt concentration or by the addition of other salts, so called elution salts, to the buffer solution. Depending on the elution method the buffer/salt concentration is either increased at once (step elution method) or continuously (continuous elution method) by the fractional addition of a concentrated buffer or elution salt solution. Preferred elution salts are sodium citrate, sodium chloride, sodium sulphate, sodium phosphate, potassium chloride, potassium sulfate, potassium phosphate, or other salts of citric acid or phosphoric acid, or any mixture of these components. In one embodiment the elution salt is sodium citrate, sodium chloride, potassium chloride, or mixtures thereof.

In some embodiments, the yield of the collected PEGylated protein after the present methods is increased at least about 1.5 fold, at least about 2 fold, at least about 3 fold, at least about 4 fold, at least about 5 fold, at least about 6 fold, at least about 7 fold, at least about 8 fold, at least about 9 fold, at least about 10 fold, at least about 20 fold, at least about 30 fold, or at least about 40 fold.

In some embodiments, the loading of the PEGylated protein having a high concentration, e.g., at least about 6 g/L, at least about 10 g/L, at least about 15 g/L, or at least about 30 g/L, results in an increase of the ion exchange matrix's loading capacity compared to the ion exchange matrix's loading capacity when PEGylated protein is loaded at a concentration lower than 6 g/L, e.g., about 5 g/L, about 4 g/L, about 3 g/L, about 2 g/L, or about 1 g/L.

In some embodiments, the loading capacity of the ion exchange matrix is increased from about 6 g to about 7 g, about 8 g, about 9 g, about 10 g, about 11 g, about 12 g, about 13 g, about 14 g, about 15 g, about 16 g, about 17 g, about 18 g, about 19 g, or about 20 g of PEGylated protein/L of matrix. In some embodiments, the loading capacity ion exchange is increased from about 6.5 g to 10 g of PEGylated protein/L of matrix. In some embodiments, the loading capacity ion exchange is increased from about 6.5 g to 11 mg of PEGylated protein/L of matrix. In some embodiments, the loading capacity ion exchange is increased from about 6.5 g to 12 g of PEGylated protein/L of matrix. In some embodiments, the loading capacity ion exchange is increased from about 6.5 g to 13 g of PEGylated protein/L of matrix. In some embodiments, the loading capacity ion exchange is increased from about 6.5 g to 14 g of PEGylated protein/L of matrix. In some embodiments, the loading capacity ion exchange is increased from about 6.5 g to 15 g of PEGylated protein/L of matrix.

In some embodiments, loading of the PEGylated protein having a high concentration, e.g., at least 6 g/L, at least about 10 g/L, at least about 15 g/L, or at least about 30 g/L, results in an increase of the ion exchange matrix's binding capacity compared to the ion exchange matrix's binding capacity when PEGylated protein is loaded at a concentration lower than 6 g/L, e.g., about 1 g/L.

In some embodiments, the binding capacity of the ion exchange matrix when loading a high concentration of PEGylated protein is increased about 1.1 fold, about 1.2 fold, about 1.3 fold, about 1.5 fold, about 1.6 fold, about 1.7 fold, about 1.8 fold, about 1.9 fold, about 2 fold, about 3 fold, about 4 fold, about 5 fold, about 6 fold, about 7 fold, about 8 fold, about 9 fold, about 10 fold, about 15 fold, about 20 fold, or about 30 fold compared to the ion exchange matrix's binding capacity when PEGylated protein is loaded at a concentration lower than 6 g/L, e.g., about 1 g/L.

In some embodiments, the binding capacity of the ion exchange chromatography matrix when loading a high concentration of PEGylated protein is at least 7 g, at least 7.5 g, at least 8 g, at least 8.5 g, at least 9 g, at least 9.5 g, at least 10 g, at least 10.5 g, at least 11 g, at least 11.5 g, at least 12 g, at least 12.5 g, at least 13 g, at least 13.5 g, at least 14 g, at least 14.5 g, at least 15 g, at least 15.5 g, at least 16 g, at least 16.5 g, or at least 17 g of PEGylated protein/L of matrix. In some embodiments, the binding capacity of the ion exchange chromatography matrix when loading a high concentration of PEGylated protein is at least 8 g of PEGylated protein/L of matrix. In some embodiments, the binding capacity of the ion exchange chromatography matrix is at least 9 g of PEGylated protein/L of matrix. In some embodiments, the binding capacity of the ion exchange chromatography matrix is at least 10 g of PEGylated protein/L of matrix. In some embodiments, the binding capacity of the ion exchange chromatography matrix is at least 11 g of PEGylated protein/L of matrix. In some embodiments, the binding capacity of the ion exchange chromatography matrix is at least 12 g of PEGylated protein/L of matrix. In some embodiments, the binding capacity of the ion exchange chromatography matrix is at least 13 g of PEGylated protein/L of matrix. In some embodiments, the binding capacity of the ion exchange chromatography matrix is at least 14 g of PEGylated protein/L of matrix. In some embodiments, the binding capacity of the ion exchange chromatography matrix is at least 15 g of PEGylated protein/L of matrix. In some embodiments, the binding capacity of the ion exchange chromatography matrix is at least 16 g of PEGylated protein/L of matrix. In some embodiments, the binding capacity of the ion exchange chromatography matrix is at least 17 g of PEGylated protein/L of matrix.

In some embodiments, the collected PEGylated protein after the ion exchange chromatography when loading a high concentration of PEGylated protein is at least about 20% pure, at least about 25% pure, at least about 30% pure, at least about 35% pure, at least about 40% pure, at least about 45% pure, at least about 50% pure, at least about 55% pure, at least about 60% pure, at least about 65% pure, at least about 70% pure, at least about 75% pure, at least about 80% pure, at least about 85% pure, at least about 90% pure, at least about 95% pure, or at least about 98% pure.

In some embodiments, UV interference during ion exchange chromatography is reduced at least at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least 98%. In some embodiments, UV interference is reduced from about 5 to about 25%. In some embodiments, UV interference is reduced about from 10 to about 30%. In some embodiments, UV interference is reduced from about 20 to about 50%. In some embodiments, UV interference is reduced from about 30 to about 60%. In some embodiments, UV interference is reduced from about 40 to about 75%. In some embodiments, UV interference is reduced from about 50 to about 80%. In some embodiments, UV interference is reduced from about 60 to about 85%. In some embodiments, UV interference is reduced from about 70 to about 90%. In some embodiments, UV interference is reduced from about 85 to about 95%. In some embodiments, UV interference is reduced from about 85 to about 99%.

In some embodiments, the loaded PEGylated protein has been concentrated without a catalyst prior to the loading. In some embodiments, a catalyst is 4-aminobenzohydrazide, 4ABH or its derivative or degraded form. Concentration is a simple process that involves removing fluid from a solution while retaining the solute molecules. The concentration of the solute increases in direct proportion to the decrease in solution volume (i.e., halving the volume effectively doubles the concentration).

In some embodiments, the loaded PEGylated protein has been concentrated by a tangential flow filtration prior to the loading. Tangential flow filtration is an ultra filtration procedure that relies on the use of fluid pressure to drive the migration of the smaller molecules through an ultrafiltration membrane while simultaneously retaining larger molecules. In general, a membrane with a molecular weight cut-off (MWCO) is selected that is three to six times smaller than the molecular weight of the protein to be retained. Other factors known to a person in the art can also impact the selection of the appropriate MWCO, e.g. flow rate, processing time, transmembrane pressure, molecular shape or structure, solute concentration, presence of other solutes, and ionic conditions. The primary applications for TFF are concentration, diafiltration (desalting and buffer exchange), and fractionation of large from small biomolecules.

Diafiltration is the fractionation process that washes smaller molecules through a membrane and leaves larger molecules in the retentate without ultimately changing concentration. It can be used to remove salts or exchange buffers. It can remove ethanol or other small solvents or additives.

Diafiltration can be continuous or discontinuous. In continuous diafiltration, the diafiltration solution (water or buffer) is added to the sample feed reservoir at the same rate as filtrate is generated. In this way, the volume in the sample reservoir remains constant, but the small molecules (e.g., salts) that can freely permeate through the membrane are washed away. Using salt removal as an example, each additional diafiltration volume (DV) reduces the salt concentration further. (One diafiltration volume is equal to adding a volume of water or buffer to the feed reservoir equal to the volume of product in the system, then concentrating back to the starting volume. For example, if you have a 200 mL sample to start, 1 DV=200 mL.) Using 2 DV will reduce the ionic strength by ˜99% with continuous diafiltration.

In discontinuous diafiltration, the solution is first diluted and then concentrated back to the starting volume. The process is then repeated until the required concentration of small molecules (e.g., salts) remaining in the reservoir is reached. Each additional DV reduces the salt concentration further. Continuous diafiltration requires less filtrate volume to achieve the same degree of salt reduction as discontinuous diafiltration. By first concentrating a sample, the amount of diafiltration solution required to achieve a specified ionic strength can be substantially reduced.

In some embodiments, the concentration of a PEGylated protein after a tangential flow filtration before the ion exchange chromatography is at least about 20 g/L, at least about 25 g/L, at least about 26 g/L, at least about 27 g/L, at least about 28 g/L, at least about 29 g/L, at least about 30 g/L, at least about 31 g/L, at least about 32 g/L, at least about 33 g/L, at least about 34 g/L, at least about 35 g/L, at least about 36 g/L, at least about 37 g/L, at least about 38 g/L, at least about 39 g/L, at least about 40 g/L, at least about 41 g/L, at least about 42 g/L, at least about 43 g/L, at least about 44 g/L, at least about 45 g/L, or at least about 50 g/L. In some embodiments, the concentration of a PEGylated protein after a tangential flow filtration before the ion exchange chromatography is at least about 35 g/L.

In some embodiments, the PEGylated protein loaded at a high concentration according to the present disclosure has a concentration of at least about 7 g/L, at least about 8 g/L, at least about 9 g/L, at least about 10 g/L, at least about 11 g/L, at least about 12 g/L, at least about 13 g/L, at least about 14 g/L, at least about 15 g/L, at least about 20 g/L, at least about 25 g/L, at least about 30 g/L, at least about 35 g/L, at least about 40 g/L, at least about 45 g/L, at least about 50 g/L, at least about 55 g/L, or at least about 60 g/L. In some embodiments, the PEGylated protein loaded at a high concentration according to the present disclosure has a concentration of from about 6 g/L to about 60 g/L, from about 10 g/L to about 60 g/L, from about 15 g/L to about 50 g/L, from about 15 g/L to about 40 g/L, from about 15 g/L to about 35 g/L, from about 15 g/L to about 40 g/L, from about 20 g/L to about 60 g/L, from about 20 g/L to about 50 g/L, from about 20 g/L to about 40 g/L, from about 20 g/L to about 35 g/L, from about 20 g/L to about 30 g/L, from about 25 g/L to about 60 g/L, from about 25 g/L to about 50 g/L, from about 25 g/L to about 40 g/L, from about 25 g/L to about 35 g/L, from about 25 g/L to about 30 g/L, or from about 30 g/L to about 35 g/L.

In some embodiments, the high concentration of the PEGylated protein loaded to an ion exchange chromatography is about 10 g/L, about 15 g/L, about 20 g/L, about 25 g/L, about 30 g/L, about 35 g/L, about 40 g/L, about 45 g/L, about 50 g/L, about 55 g/L, or about 60 g/L.

In some embodiments, the PEGylated protein loaded at a high concentration has a concentration of about 15 g/L. In some embodiments, the PEGylated protein loaded at a high concentration has a concentration of about 20 g/L. In some embodiments, the PEGylated protein loaded at a high concentration has a concentration of about 25 g/L. In some embodiments, the PEGylated protein loaded at a high concentration has a concentration of about 30 g/L. In some embodiments, the PEGylated protein loaded at a high concentration has a concentration of about 35 g/L.

In some embodiments, the protein yield of the PEGylated protein after running the ion exchange chromatography according to the present disclosure is increased at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least 70%, at least about 75%, at least about 80%, at least about 85%, at least about 90%, at least about 95%, at least about 98%, or at least about 99% compared to the ion exchange matrix's binding capacity when PEGylated protein is loaded at a concentration lower than 6 g/L, e.g., about 1 g/L. In some embodiments, the protein yield of the PEGylated protein after running the ion exchange chromatography according to the present disclosure is increased from about 10% to about 20%. In some embodiments, the protein yield of the PEGylated protein after running the ion exchange chromatography according to the present disclosure is increased from about 15% to about 30% compared to the ion exchange matrix's binding capacity when PEGylated protein is loaded at a concentration lower than 6 g/L, e.g., about 1 g/L. In some embodiments, the protein yield of the PEGylated protein after running the ion exchange chromatography according to the present disclosure is increased from about 20% to about 35% compared to the ion exchange matrix's binding capacity when PEGylated protein is loaded at a concentration lower than 6 g/L, e.g., about 1 g/L. In some embodiments, the protein yield of the PEGylated protein after running the ion exchange chromatography according to the present disclosure is increased from about 25% to about 40% compared to the ion exchange matrix's binding capacity when PEGylated protein is loaded at a concentration lower than 6 g/L, e.g., about 1 g/L. In some embodiments, the protein yield of the PEGylated protein after running the ion exchange chromatography according to the present disclosure is increased from about 45% to about 60% compared to the ion exchange matrix's binding capacity when PEGylated protein is loaded at a concentration lower than 6 g/L, e.g., about 1 g/L. In some embodiments, the protein yield of the PEGylated protein after running the ion exchange chromatography according to the present disclosure is increased from about 65% to about 80% compared to the ion exchange matrix's binding capacity when PEGylated protein is loaded at a concentration lower than 6 g/L, e.g., about 1 g/L. In some embodiments, the protein yield of the PEGylated protein after running the ion exchange chromatography according to the present disclosure is increased from about 85% to about 90% compared to the ion exchange matrix's binding capacity when PEGylated protein is loaded at a concentration lower than 6 g/L, e.g., about 1 g/L. In some embodiments, the protein yield of the PEGylated protein after running the ion exchange chromatography according to the present disclosure is increased from about 90% to about 99% compared to the ion exchange matrix's binding capacity when PEGylated protein is loaded at a concentration lower than 6 g/L, e.g., about 1 g/L. In some embodiments, the method further comprises washing the matrix using a wash buffer. Buffer pH and ionic strength are crucial for all forms of ion exchange chromatography. Buffer counterions should have the same charge as the resin; Tris buffers are generally used for positively charged anion exchange resins, and phosphate buffers are generally used for negatively charged cation exchange resins.

In some embodiments, the method further comprises eluting the PEGylated protein using an elution buffer. The elution buffer is designed to recover or collect the polypeptide bound to the ion exchange material. Generally, the ionic strength, i.e., the conductivity, of the buffer/solution passing through the ion exchange column is increased. This can be accomplished either by an increased buffer salt concentration or by the addition of other salts, so called elution salts, to the buffer solution. Depending on the elution method the buffer/salt concentration is either increased at once (step elution method) or continuously (continuous elution method) by the fractional addition of a concentrated buffer or elution salt solution. Preferred elution salts are sodium citrate, sodium chloride, sodium sulphate, sodium phosphate, potassium chloride, potassium sulfate, potassium phosphate, or other salts of citric acid or phosphoric acid, or any mixture of these components. In one embodiment the elution salt is sodium citrate, sodium chloride, potassium chloride, or mixtures thereof.

In some embodiments, the ion exchange chromatography is a cation exchange chromatography. Cation exchange chromatography uses a negatively charged ion exchange resin with an affinity for molecules having net positive surface charges. Cation exchange chromatography is used both for preparative and analytical purposes and can separate a large range of molecules from amino acids and nucleotides to large proteins.

In some embodiments, the ion exchange chromatography comprises a CEX resin selected from the group consisting of Poros HS, Poros XS, carboxy-methyl-cellulose, BAKERBOND ABX™, sulphopropyl immobilized on agarose and sulphonyl immobilized on agarose, MonoS, MiniS, Source 15S, 30S, SP SEPHAROSE™, CM SEPHAROSE™ BAKERBOND Carboxy-Sulfon, WP CBX, WP Sulfonic, Hydrocell CM, Hydrocel SP, UNOsphere S, Macro-Prep High S, Macro-Prep CM, Ceramic HyperD S, Ceramic HyperD CM, Ceramic HyperD Z, Trisacryl M CM, Trisacryl LS CM, Trisacryl M SP, Trisacryl LS SP, Spherodex LS SP, DOWEX Fine Mesh Strong Acid Cation Resin, DOWEX MAC-3, Matrex Cellufine C500, Matrex Cellufine C200, Fractogel EMD S03-, Fractogel EMD SE, Fractogel EMD COO—, Amberlite Weak and Strong Cation Exchangers, Diaion Weak and Strong Cation Exchangers, TSK Gel SP-5PW-HR, TSK Gel SP-5PW, Toyopearl CM (650S, 650M, 650C), Toyopearl SP (650S, 650M, 650C), CM (23, 32, 52), SE(52, 53), P11, Express-Ion C and Express-Ion S, and any combination thereof.

In some embodiments, the ion exchange chromatography is an anion exchange chromatography. Anion exchange chromatography uses a positively charged ion exchange resin with an affinity for molecules having net negative surface charges. Anion exchange chromatography is used both for preparative and analytical purposes and can separate a large range of molecules, from amino acids and nucleotides to large proteins.

In some embodiments, the anion exchange chromatography comprises a AEX resin selected from the group consisting of POROS HQ, POROS XQ, Q SEPHAROSE™ Fast Flow, DEAE SEPHAROSE™ Fast Flow, SARTOBIND® Q, ANX SEPHAROSE™ 4 Fast Flow (high sub), Q SEPHAROSE™ XL, Q SEPHAROSE™ big beads, DEAE Sephadex A-25, DEAE Sephadex A-50, QAE Sephadex A-25, QAE Sephadex A-50, Q SEPHAROSE™ high performance, Q SEPHAROSE™ XL, Sourse 15Q, Sourse 30Q, Resourse Q, Capto Q, Capto DEAE, Mono Q, Toyopearl Super Q, Toyopearl DEAE, Toyopearl QAE, Toyopearl Q, Toyopearl GigaCap Q, TS gel SuperQ, TS gel DEAE, Fractogel EMD TMAE, Fractogel EMD TMAE HiCap, Fractogel EMD DEAE, Fractogel EMD DMAE, Macroprep High Q, Macro-prep-DEAE, Unosphere Q, Nuvia Q, PORGS PI, DEAE Ceramic HyperD, Q Ceramic HyperD, and any combination thereof.

In some embodiments, the PEGylated protein useful for the present disclosure has a molecular weight of at least about 5, at least about 10 kDa, at least about 15 kDa, at least about 20 kDa, at least about 25 kDa, at least about 30 kDa, at least about 35 kDa, at least about 40 kDa, at least about 45 kDa, at least about 50 kDa, at least about 55 kDa, at least about 60 kDa, at least about 75 kDa, at least about 80 kDa, at least about 85 kDa, at least about 90 kDa, at least about 95 kDa, at least about 100 kDa, at least about 105 kDa, at least about 110 kDa, at least about 115 kDa, at least about 120 kDa, at least about 125 kDa, at least about 130 kDa, at least about 135 kDa, at least about 140 kDa, at least about 145 kDa, at least about 150 kDa, at least about 155 kDa, at least about 160 kDa, at least about 165 kDa, at least about 170 kDa, at least about 175 kDa, at least about 180 kDa, at least about 185 kDa, at least about 190 kDa, at least about 195 kDa, at least about 200 kDa, at least about 300 kDa, at least about 35 kDa 0, at least about 400 kDa, at least about 450 kDa, at least about 500 kDa, at least about 550 kDa, or at least about 600 kDa. In some embodiments, the PEGylated protein useful for the present disclosure has a molecular weight of about 2 kDa to about 15 kDa. In some embodiments, the PEGylated protein useful for the present disclosure has a molecular weight of about 15 kDa to about 35 kDa. In some embodiments, the PEGylated protein useful for the present disclosure has a molecular weight of about 35 kDa to about 55 kDa. In some embodiments, the PEGylated protein useful for the present disclosure has a molecular weight of about 50 kDa to about 75 kDa. In some embodiments, the PEGylated protein useful for the present disclosure has a molecular weight of about 70 kDa to about 95 kDa. In some embodiments, the PEGylated protein useful for the present disclosure has a molecular weight of about 80 kDa to about 115 kDa. In some embodiments, the PEGylated protein useful for the present disclosure has a molecular weight of about 95 kDa to about 140 kDa. In some embodiments, the PEGylated protein useful for the present disclosure has a molecular weight of about 135 kDa to about 170 kDa. In some embodiments, the PEGylated protein useful for the present disclosure has a molecular weight of about 160 kDa to about 200 kDa. In some embodiments, the PEGylated protein useful for the present disclosure has a molecular weight of about 180 kDa to about 235 kDa. In some embodiments, the PEGylated protein useful for the present disclosure has a molecular weight of about 205 kDa to about 250 kDa. In some embodiments, the PEGylated protein useful for the present disclosure has a molecular weight of about 225 kDa to about 280 kDa. In some embodiments, the PEGylated protein useful for the present disclosure has a molecular weight of about 270 kDa to about 330 kDa. In some embodiments, the PEGylated protein useful for the present disclosure has a molecular weight of about 325 kDa to about 360 kDa. In some embodiments, the PEGylated protein useful for the present disclosure has a molecular weight of about 350 kDa to about 425 kDa. In some embodiments, the PEGylated protein useful for the present disclosure has a molecular weight of about 415 kDa to about 465 kDa. In some embodiments, the PEGylated protein useful for the present disclosure has a molecular weight of about 450 kDa to about 500 kDa. In some embodiments, the PEGylated protein useful for the present disclosure has a molecular weight of about 485 kDa to about 525 kDa. In some embodiments, the PEGylated protein useful for the present disclosure has a molecular weight of about 500 kDa to about 550 kDa. In some embodiments, the PEGylated protein useful for the present disclosure has a molecular weight of about 530 kDa to about 575 kDa. In some embodiments, the PEGylated protein useful for the present disclosure has a molecular weight of about 560 kDa to about 605 kDa.

In some embodiments, the PEGylated protein useful for the present disclosure is a wild-type protein, a mutant, a derivative, a variant, or a fragment that has been PEGylated, wherein protein origin can be mammalian, eukaryotic or prokaryotic origin, including but not limited to, for example growth factors, e.g., FGF21, human granulocyte colony-stimulating factor; interleukins, e.g., interleukin 2; blood clotting factors, e.g., Factor VIII or IX; interferons, e.g., interferon alfa 1a, interferon alfa 1b, interferon alfa 2b, interferon beta 1; opioid antagonists; hormones, e.g., erythropoietin; hormone antagonists, e.g., human growth hormone antagonist; enzymes, e.g., L-asparaginase, adenosine deaminase, uricase, hyaluronidase; antibodies, e.g., Fab1 or Fab2 fragment of an antibody, e.g., Fab fragment of a monoclonal antibody to human tumor necrosis factor alpha (TNFα); cytokines, e.g., IL10; proteins encapsulated in PEGylated liposomes, e.g., doxorubicin; and antibiotics.

In some embodiments, the PEGylated protein useful for the present disclosure is a naturally occurring or recombinantly produced protein, or a fusion protein that has been PEGylated.

In some embodiments, the PEGylated protein useful for the present disclosure is an antibody wherein the protein origin can be mammalian, eukaryotic or prokaryotic origin, including but not limited to a polyclonal antibody, a monoclonal antibody, a humanized antibody, a bispecific antibody, a multispecific antibody, an IgA, IgG or IgM antibody, an antigen binding portion of an antibody, e.g., a Fab1 or Fab2 fragment of an antibody, e.g., Fab fragment of a monoclonal antibody to human tumor necrosis factor alpha (TNFα), a Fd fragment consisting of the VH and CH1 domains, a Fv fragment consisting of the VL and VH domains of a single arm of an antibody, a dAb fragment which consists of a VH domain, an isolated complementarity determining region (CDR) and a combination of two or more isolated CDRs which can optionally be joined by a synthetic linker.

In some embodiments, the PEGylated protein useful for the present disclosure is a cytokine, a clotting factor, a hormone, a cell surface receptor, a growth factor, or any combination thereof.

In some embodiments, the PEGylated protein is fibroblast growth factor 21 FGF21 wild-type or modified FGF-21 polypeptide. As used herein, “modified FGF-21 polypeptide,” shall include those polypeptides and proteins that differ from wild-type FGF-21 and typically have at least one biological activity of a fibroblast growth factor 21, as well as FGF-21 analogs, FGF-21 isoforms, FGF-21 mimetics, FGF-21 fragments, hybrid FGF-21 proteins, fusion proteins, oligomers and multimers, homologues, glycosylation pattern variants, variants, splice variants, and muteins thereof, regardless of the biological activity of the same. Certain FGF-21 polypeptides and uses thereof are described in U.S. Patent Publication No. 20010012628, U.S. Pat. No. 6,716,626, U.S. Patent Publication No. 2004/0259780, WO 03/011213, Kharitonenkov et al. J Clin Invest. 2005 June; 115(6): 1627-35, WO 03/059270, U.S. Patent Publication No. 2005/0176631, WO 2005/091944, WO 2007/0293430, U.S. Patent Publication No. 2007/0293430, WO/2008/121563, U.S. Pat. No. 4,904,584, WO 99/67291, WO 99/03887, WO 00/26354, and U.S. Pat. No. 5,218,092 each of which is incorporated by reference herein in its entirety.

In some embodiments, the PEGylated protein comprises a PEGylation moiety.

In some embodiments, the PEGylation moiety is linear, branched, mono-PEGylated, random PEGylated, and multiple PEGylated (PEGmers).

In some embodiments, the PEGylation moiety is at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 40, at least about 50, at least about 55, at least about 60, at least about 65, at least about 70, at least about 75, at least about 80, at least about 90, at least about 95, at least about 100, kDa. In some embodiments, the PEGylation moiety is from about 1 to about 100 kDa. In some embodiments, the PEGylation moiety is from about 2 to about 5 kDa. In some embodiments, the PEGylation moiety is from about 10 to about 20 kDa. In some embodiments, the PEGylation moiety is from about 25 to about 50 kDa. In some embodiments, the PEGylation moiety is from about 2 to about 50 kDa. In some embodiments, the PEGylation moiety is from about 20 to about 100 kDa. In some embodiments, the PEGylation moiety is from about 5 to about 30 kDa. In some embodiments, the PEGylation moiety is from about 5 to about 40 kDa. In some embodiments, the PEGylation moiety is from about 10 to about 80 kDa.

In some embodiments, the PEGylation moiety is about 30 kDa.

In some embodiments, a purified PEGylated protein using the method of the present disclosure is, for example, Fibroblast Growth Factor 21 (FGF21), Interleukin 2, Factor VIII, Factor IX, recombinant phenylalanine ammonia-lyase, an interferon (e.g., Interferon Beta-1a), an opioid antagonist such as naloxol, Certolizumab pegol, erythropoietin (e.g., methoxy polyethylene glycol-epoetin beta), Pegaptanib, a recombinant methionyl human granulocyte colony-stimulating factor, Pegfilgrastim, a human growth hormone antagonist (e.g., Pegvisomant), interferon alpha, (e.g., Peginterferon alfa-2a or Peginterferon alfa-2b), L-asparaginase (e.g., Pegaspargase), adenosine deaminase (e.g., Pegademase bovine, Adagen), PEG-uricase, pegloticase, an enzyme that metabolizes uric acid (Krystexxa), recombinant human hyaluronidase, asparaginase, a humanized antibody such as alacizumab, a Fab fragment of a monoclonal antibody such as Certolizumab, soluble tumor necrosis factor (Pegsunercept), interleukins such as recombinant murine IL-10, doxorubicin, to name a few.

Examples of PEGylated proteins include, but are not limited to, the following:
PALYNZIQ®—PEGylated recombinant phenylalanine ammonia-lyase for the treatment of Phenylketonuria, approved by the FDA for the US in May 2018 (BioMarin).
ADYNOVATE®—Recombinant PEGylated Antihemophilic Factor VIII for the treatment of patients with hemophilia A. (Baxalta, 2015)
PLEGRIDY®— PEGylated Interferon Beta-1a for the treatment of patients with relapsing forms of multiple sclerosis. (Biogen, 2014)
MOVANTIK® (Naloxegol)—PEGylated naloxol for the treatment of opioid-induced constipation in adults patients with chronic non-cancer pain (un-pegylated methadone can cause adverse gastrointestinal reactions). (AstraZeneca, 2014)
OMONTYA® (Peginesatide)—once-monthly medication to treat anemia associated with chronic kidney disease in adult patients on dialysis (Affymax/Takeda Pharmaceuticals, 2012)
KRYSTEXXA® (Pegloticase)—PEGylated uricase for the treatment of gout (Savirnt, 2010)
CYMZIA® (Certolizumab pegol)—monoclonal antibody for treatment of moderate to severe rheumatoid arthritis and Crohn's disease, an inflammatory gastrointestinal disorder (Nektar/UCB Pharma, 2008)
MIRCERA® (Methoxy polyethylene glycol-epoetin beta)—PEGylated form of erythropoietin to combat anemia associated with chronic kidney disease (Roche, 2007)
MACUGEN® (Pegaptanib)—used to treat neovascular age-related macular degeneration (Pfizer, 2004)
NEULASTA® (Pegfilgrastim)—PEGylated recombinant methionyl human granulocyte colony-stimulating factor for severe cancer chemotherapy-induced neutropenia (Amgen, 2002)
SOMAVERT® (Pegvisomant)—PEG-human growth hormone mutein antagonist for treatment of Acromegaly (Pfizer, 2002)
PEGASYS® (Peginterferon alfa-2a)—PEGylated interferon alpha for use in the treatment of chronic hepatitis C and hepatitis B (Hoffmann-La Roche, 2002)
PEGINTRON® (Peginterferon alfa-2b)—PEGylated interferon alpha for use in the treatment of chronic hepatitis C and hepatitis B (Schering-Plough/Enzon, 2000)
DOXIL®/CAELYX® (Doxorubicin HCl liposome)—PEGylated liposome containing doxorubicin for the treatment of cancer (Alza 1995)
MYOCET® (Doxorubicin HCl liposome)—PEGylated liposome containing doxorubicin for the treatment of cancer (Teva UK)
ONCASPAR® (Pegaspargase)—PEGylated L-asparaginase for the treatment of acute lymphoblastic leukemia in patients who are hypersensitive to the native unmodified form of L-asparaginase (Enzon, 1994). This drug is also approved for front line use.
ADAGEN® (Pegademase bovine)—PEG-adenosine deaminase for the treatment of severe combined immunodeficiency disease (SCID) (Enzon, 1990)

### III. Composition and Methods of Treating

The present disclosure also includes a protein purified according to the present methods. In some embodiments, the purified proteins can further be formulated to be suitable for administering in mammal, e.g., human. In other embodiments, the present disclosure includes a method of treating or preventing a disease or condition comprising administering the protein purified by the present methods.

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

The present disclosure has been described above and below with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The description of the specific embodiments herein will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the exemplary embodiments described herein, but should be defined only in accordance with the following claims and their equivalents.

### EXAMPLES

### Example 1. Impact of Protein Concentration on the Size of PEGylated Proteins and their Adsorption in Ion Exchange Chromatography

Proteins of all sizes can be PEGylated to improve pharmacokinetics profiles for therapeutic purpose. PEGylated proteins present a few challenges during the downstream processing. Ion exchange chromatography is used for purification of PEGylated proteins. However, the dynamic binding capacity of PEGylated proteins is significantly reduced compared with the native proteins. The potential causes include the shielding of protein charge by conjugated PEG polymer chains and reduced diffusivity in resin beads due to large PEGylated protein sizes.

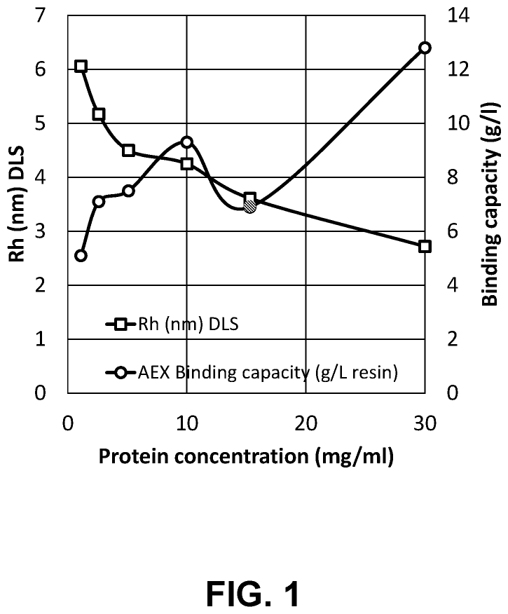

In this study, we found that PEGylated-protein concentration can impact the size and structure of PEGylated protein and thus its binding behavior in ion exchange chromatography (see FIG. 1). Another challenge in connection with PEGylation reactions is that the PEGylation reaction catalyst's UV absorbance can interfere with the subsequent chromatogram and with protein concentration determinations.

We utilized tangential flow filtration to concentrate PEGylated proteins and remove the catalyst and other impurities. Tangential Flow Filtration was performed by initial dilution of the PEGylation mixture, diafiltration with AEX loading buffer and final concentration for AEX loading. AEX was performed by column equilibration, protein loading, column wash, and elution using a linear gradient, followed by strip and cleaning. With diafiltrated and concentrated PEGylated proteins, a higher binding and loading capacity was achieved and UV interference was removed in ion exchange chromatography (see FIG. 1).

The binding capacity of the AEX resin for concentrated PEGylated FGF21 and the hydrodynamic radius of PEGylated FGF21 were determined as a function of the PEGylated FGF21 concentration loaded onto the AEX resin (see FIG. 1). FGF21 was PEGylated according to methods known in the art.

The hydrodynamic radius was determined using dynamic light scattering, which is a method commonly used in the art. Dynamic binding capacity was determined by loading protein on the AEX column and monitoring the bound protein before breakthrough by UV280, which is a method commonly used in the art.

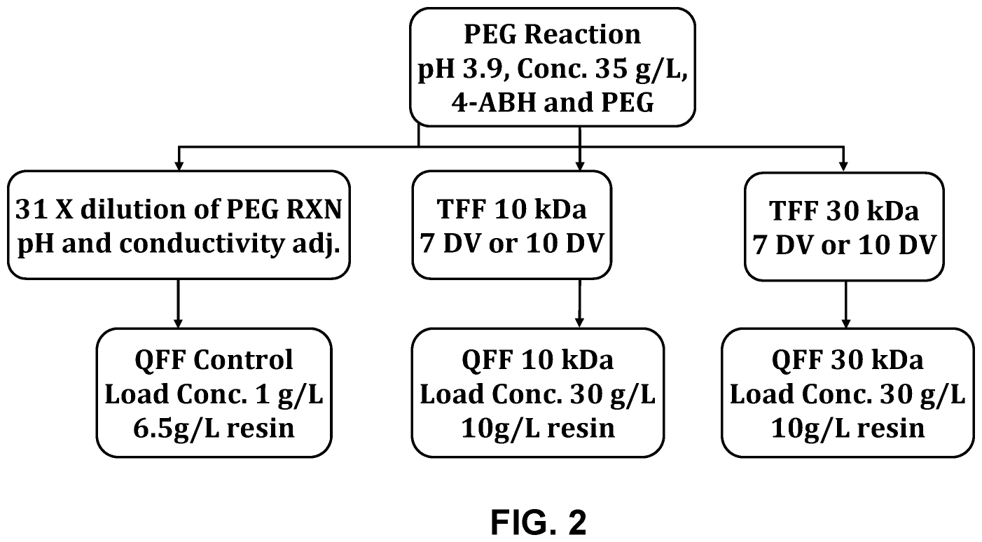

As shown in FIG. 2, the current process in which the PEGylation reaction was diluted 31× and then subjected to anion exchange chromatography at a loading concentration of 1 g of PEGylated FGF21/L resulted in a binding capacity of 6.5 g PEGylated FGF21/L of AEX resin. By contrast, in case the PEGylated FGF21 was concentrated by tangential flow filtration (either having a pore size of 10 kDa or 30 kDa as indicated) to a loading concentration of 30 g of PEGylated FGF21/L, the resulting binding capacity was 10 g of PEGylated FGF21/L of AEX resin.

Abbreviations: Rh, Hydrodynamic radius; DLS, dynamic light scattering; DBC, Dynamic binding capacity; AEX, anion exchange chromatography; TFF, tangential flow filtration; DV, diafiltration volume; QFF, Q Sepharose Fast Flow; 4ABH, 4-aminobenzoic acid hydrazide; PABA, p-aminobenzoic acid.

What is claimed is:

1. A method for purifying a PEGylated protein, comprising loading PEGylated protein having a high concentration of at least 6 grams/liter on an ion exchange chromatography matrix, and collecting the PEGylated protein.

2. The method of claim 1, wherein the loading of the PEGylated protein having a high concentration results in an increase in the yield of the collected PEGylated protein compared to the yield of the collected PEGylated protein loaded at the concentration of 1 g/L.

3. The method of claim 2, wherein the yield of the collected PEGylated protein is increased at least about 2 fold, about 3 fold, about 4 fold, about 5 fold, about 6 fold, about 7 fold, about 8 fold, about 9 fold, about 10 fold, about 20 fold, about 30 fold, or about 40 fold.

4. The method of any one of claims 1 to 3, wherein the loading of the PEGylated protein having a high concentration results in an increase of the ion exchange chromatography matrix's loading capacity compared to the ion exchange matrix's loading capacity when PEGylated protein is loaded at a concentration of 1 g/L.

5. The method of claim 4, wherein the loading capacity of the ion exchange matrix is increased from 6 to 7, 8, 9, 10, 11, 12, 13, 14, 15, 16, 17, 18, 19 and 20 g of PEGylated protein/L of matrix.

6. The method of any one of claims 1 to 5, wherein the loading of the PEGylated protein having a high concentration results in an increase of the ion exchange chromatography matrix's binding capacity compared to the ion exchange chromatography matrix's binding capacity when PEGylated protein is loaded at a concentration of 1 g/L.

7. The method of claim 6, wherein the binding capacity of the ion exchange matrix is increased about 1.1 fold, about 1.2 fold, about 1.3 fold, about 1.5 fold, about 1.6 fold, about 1.7 fold, about 1.8 fold, about 1.9 fold, about 2 fold, about 3 fold, about 4 fold, about 5 fold, about 6 fold, about 7 fold, about 8 fold, about 9 fold, about 10 fold, about 15 fold, about 20 fold, or about 30 fold.

8. The method of claim 6 or 7, wherein the binding capacity of the ion exchange chromatography matrix is at least 7, at least 7.5, at least 8, at least 8.5, at least 9, at least 9.5, at least 10, at least 10.5, at least 11, at least 11.5, at least 12, at least 12.5, at least 13, at least 13.5, at least 14, at least 14.5, at last 15, at least 15.5, at least 16, at least 16.5, or at least 17 g of PEGylated protein//L of matrix.

9. The method of any one of claims 1 to 8, wherein the collected PEGylated protein is at least about 20% pure, at least about 25% pure, at least about 30% pure, at least about 35% pure, at least about 40% pure, at least about 45% pure, at least about 50% pure, at least about 55% pure, at least about 60% pure, at least about 65% pure, at least about 70% pure, at least about 75% pure, at least about 80% pure, at least about 85% pure, at least about 90% pure, at least about 95% pure, at least about 98% pure.

10. The method of any one of claims 1 to 9, wherein UV interference during ion exchange chromatography is reduced at least at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least 98%.

11. The method of any one of claims 1 to 10, wherein the loaded PEGylated protein has been concentrated without a catalyst prior to the loading.

12. The method of any one of claims 1 to 11, wherein the loaded PEGylated protein has been concentrated by a tangential flow filtration prior to the loading.

13. The method of any one of claims 2 to 12, wherein the PEGylated protein loaded at a high concentration has a concentration of at least about 10, at least about 15, at least about 20, at least about 25, at least about 30, at least about 35, at least about 40, at least about 45, at least about 50, at least about 55, at least about 60 g/L.

14. The method of claim 13, wherein the PEGylated protein loaded at a high concentration has a concentration of about 10, about 15, about 20, about 25, about 30, about 35, about 40, about 45, about 50, about 55, about 60 g/L.

15. The method of claim 14, wherein the PEGylated protein loaded at a high concentration has a concentration of about 30 g/L.

16. The method of claim 13, wherein the yield of the collected PEGylated protein is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99%.

17. The method of any one of claims 1 to 16, further comprising washing the matrix using a wash buffer.

18. The method of any one of claims 1 to 17, further comprising eluting the PEGylated protein using an elution buffer.

19. The method of any one of claims 1 to 18, wherein the ion exchange chromatography is a cation exchange chromatography.

20. The method of claim 19, wherein the ion exchange chromatography comprises a CEX resin selected from the group consisting of Poros HS, Poros XS, carboxy-methyl-cellulose, BAKERBOND ABX™, sulphopropyl immobilized on agarose and sulphonyl immobilized on agarose, MonoS, MiniS, Source 15S, 30S, SP SEPHAROSE™, CM SEPHAROSE™ BAKERBOND Carboxy-Sulfon, WP CBX, WP Sulfonic, Hydrocell CM, Hydrocel SP, UNOsphere S, Macro-Prep High S, Macro-Prep CM, Ceramic HyperD S, Ceramic HyperD CM, Ceramic HyperD Z, Trisacryl M CM, Trisacryl LS CM, Trisacryl M SP, Trisacryl LS SP, Spherodex LS SP, DOWEX Fine Mesh Strong Acid Cation Resin, DOWEX MAC-3, Matrex Cellufine C500, Matrex Cellufine C200, Fractogel EMD SO3-, Fractogel EMD SE, Fractogel EMD COO—, Amberlite Weak and Strong Cation Exchangers, Diaion Weak and Strong Cation Exchangers, TSK Gel SP—SPW-HR, TSK Gel SP-SPW, Toyopearl CM (650S, 650M, 650C), Toyopearl SP (650S, 650M, 650C), CM (23, 32, 52), SE(52, 53), P11, Express-Ion C and Express-Ion S, and any combination thereof.

21. The method of any one of claims 1 to 18, wherein the ion exchange chromatography is an anion exchange chromatography.

22. The method of claim 21, wherein the anion exchange chromatography comprises a AEX resin selected from the group consisting of POROS HQ, POROS XQ, Q SEPHAROSE™ Fast Flow, DEAE SEPHAROSE™ Fast Flow, SARTOBIND® Q, ANX SEPHAROSE™ 4 Fast Flow (high sub), Q SEPHAROSE™ XL, Q SEPHAROSE™ big beads, DEAE Sephadex A-25, DEAE Sephadex A-50, QAE Sephadex A-25, QAE Sephadex A-50, Q SEPHAROSE™ high performance, Q SEPHAROSE™ XL, Sourse 15Q, Sourse 30Q, Resourse Q, Capto Q, Capto DEAE, Mono Q, Toyopearl Super Q, Toyopearl DEAE, Toyopearl QAE, Toyopearl Q, Toyopearl GigaCap Q, TS gel SuperQ, TS gel DEAE, Fractogel EMD TMAE, Fractogel EMD TMAE HiCap, Fractogel EMD DEAE, Fractogel EMD DMAE, Macroprep High Q, Macro-prep-DEAE, Unosphere Q, Nuvia Q, PORGS PI, DEAE Ceramic HyperD, Q Ceramic HyperD, and any combination thereof.

23. The method of any of claims 1 to 22 wherein the PEGylated protein has a molecular weight of at least about 5, at least about 10, at least about 15, at least about 20, at least about 25, at least about 30, at least about 35, at least about 40, at least about 45, at least about 50, at least about 55, at least about 60, at least about 75, at least about 80, at least about 85, at least about 90, at least about 95, at least about 100, at least about 105, at least about 110, at least about 115, at least about 120, at least about 125, at least about 130, at least about 135, at least about 140, at least about 145, at least about 150, at least about 155, at least about 160, at least about 165, at least about 170, at least about 175, at least about 180, at least about 185, at least about 190, at least about 195, at least about 200, at least about 300, at least about 350, at least about 400, at least about 450, at least about 500, at least about 550, or at least about 600 kDa.

24. The method of any one of claims 1 to 23, wherein the PEGylated protein is a wild-type protein, a mutant, a derivative, a variant, or a fragment thereof.

25. The method of any one of claims 1 to 24, wherein the PEGylated protein is a naturally occurring or recombinantly produced protein.

26. The method of any one of claims 1 to 25, wherein the PEGylated protein is an antibody or a fusion protein.

27. The method of any one of claims 1 to 26, wherein the PEGylated protein is a cytokine, a clotting factor, a hormone, a cell surface receptor, a growth factor, or any combination thereof.

28. The method of any one of claims 1 to 27, wherein the PEGylated protein is Fibroblast Growth Factor 21 (FGF21), Interleukin 2, Factor VIII, recombinant phenylalanine ammonia-lyase, Pegvaliase, Adynovate, an interferon (e.g., Interferon Beta-1a (e.g., Plegridy)), naloxol (e.g., Naloxegol), Peginesatide, Certolizumab pegol, erythropoietin (e.g., methoxy polyethylene glycol-epoetin beta), Pegaptanib, a recombinant methionyl human granulocyte colony-stimulating factor, Pegfilgrastim, a human growth hormone antagonist (e.g., Pegvisomant), interferon alpha, (e.g., Peginterferon alfa-2a or Peginterferon alfa-2b), L-asparaginase (e.g., Pegaspargase), adenosine deaminase (e.g., Pegademase bovine), or doxorubicin.

29. The method of any one of claims 1 to 28, wherein the PEGylated protein is FGF21.

30. The method of any of claims 1 to 28, wherein the PEGylated protein comprises a PEGylation moiety.

31. The method of claim 30 wherein the PEGylation moiety is linear, branched, mono-PEGylated, random PEGylated, and multiple PEGylated (PEGmers).

32. The method of claim 31, wherein the PEGylation moiety is at least about 1 kDa, at least about 2 kDa, at least about 3 kDa, at least about 4 kDa, at least about 5 kDa, at least about 6 kDa, at least about 7 kDa, at least about 8 kDa, at least about 9 kDa, at least about 10 kDa, at least about 11 kDa, at least about 12 kDa, at least about 13 kDa, at least about 14 kDa, at least about 15 kDa, at least about 16 kDa, at least about 17 kDa, at least about 18, at least about 19, at least about 20, at least about 21, at least about 22, at least about 23, at least about 24, at least about 25, at least about 30, at least about 40, at least about 50, at least about 55, at least about 60, at least about 65, at least about 70, at least about 75, at least about 80, at least about 90, at least about 95, or at least about 100 kDa.

33. The method of claim 32, wherein the PEGylation moiety is about 30 kDa.

34. A protein purified by the method of any one of claims 1 to 33.
